(19) United States
(12) Patent Application Publication
(10) Pub. No.: US 2025/0304443 A1
(43) Pub. Date: Oct. 2, 2025

(54) SYSTEMS, METHODS, AND APPARATUS FOR PRODUCING NITRIC OXIDE

(71) Applicant: VERO Biotech Inc., Atlanta, GA (US)

(72) Inventors: Christopher Varga, Atlanta, GA (US); Kathleen Fisher, Atlanta, GA (US)

(21) Appl. No.: 19/097,163

(22) Filed: Apr. 1, 2025

Related U.S. Application Data
(60) Provisional application No. 63/573,175, filed on Apr. 2, 2024.

(51) Int. Cl. C01B 21/24; A61M 16/14; B01J 7/00

(52) U.S. Cl. CPC C01B 21/24; A61M 16/14; B01J 7/00; A61M 2202/0007; A61M 2202/0275

(57) ABSTRACT

Disclosed herein is a device comprising: a media configured to convert nitrogen dioxide into nitric oxide, wherein the media comprises: (a) a support material having a surface area of 350 to 5000 m²/g; and (b) an antioxidant material.

### CROSS-REFERENCE TO RELATED APPLICATIONS

This application claims the benefit of U.S. Provisional Application No. 63/573,175, filed on Apr. 2, 2024, the contents of which are incorporated herein by reference in their entirety.

### FIELD

Some aspects described herein relate to a medical device and, more particularly, to systems and methods for producing and delivering a gas that includes nitric oxide.

### BACKGROUND

Some aspects described herein relate to the production of nitric oxide (NO), which is then typically delivered to a patient in a medical setting.

Nitric oxide is a vasodilator indicated to improve oxygenation and reduce the need for extracorporeal membrane oxygenation, particularly in term and near-term neonates with hypoxic respiratory failure associated with clinical or echocardiographic evidence of pulmonary hypertension in conjunction with ventilatory support. Low concentrations of inhaled nitric oxide can also prevent, reverse, or limit the progression of disorders, which can include, but are not limited to, acute pulmonary vasoconstriction, traumatic injury, aspiration or inhalation injury, fat embolism in the lung, acidosis, inflammation of the lung, adult respiratory distress syndrome, acute pulmonary edema, acute mountain sickness, post cardiac surgery acute pulmonary hypertension, persistent pulmonary hypertension of a newborn, perinatal aspiration syndrome, hyaline membrane disease, acute pulmonary thromboembolism, heparin-protamine reactions, sepsis, asthma and status asthmaticus or hypoxia. Nitric oxide can also be used to treat chronic pulmonary hypertension, bronchopulmonary dysplasia, chronic pulmonary thromboembolism, and idiopathic or primary pulmonary hypertension or chronic hypoxia.

Inhaled nitric oxide therapy typically involves the delivery of nitric oxide in parts per billion to parts per million concentrations within a breathing gas, generally composing air or oxygen-enriched air. This breathing gas may contain other components, such as anesthetic agents, nebulized liquids, or other gaseous components, and it is typically conveyed to a patient using either a mechanical or manual ventilation device. In some inhaled nitric oxide delivery systems, the nitric oxide is provided within pressurized tanks, whereas in other systems, the nitric oxide may be generated on demand within the delivery system itself. One such system is described in U.S. Pat. No. 11,744,978, the content of which is incorporated herein in its entirety. In this approach, nitric oxide is produced by a chemical reaction between NO₂ gas and an antioxidant wherein the NO₂ gas is produced through a phase-change of liquid N₂O₄. In such systems, the liquid N₂O₄ is typically housed in a pressure vessel with components required for reaction control (e.g., heating and cooling components), reactant mixing, and measurement co-located with the reactants themselves. Although this is an effective approach, there is a need for a system wherein the reactants required to create nitric oxide gas for a patient are housed within a simple component, and the components that are required to initiate, contain, measure, and control the reaction reside in a location where they can be used many times. This creates the need for novel packaging, geometries, and orientations of reactants, as well as novel loading, activation, and ejection mechanisms.

This need and all other needs are at least partially addressed by this disclosure.

### SUMMARY

The present disclosure is directed to a device comprising: a media configured to convert nitrogen dioxide into nitric oxide, wherein the media comprises: (a) a support material having a specific surface area of 350 to 5000 m²/g; and (b) an antioxidant material.

In still further aspects, the support material can comprise a molecular sieve, a metal-organic framework (MOF), natural zeolites, synthetic zeolites, polymers of intrinsic microporosity (PIMs), hyper-crosslinked microporous polymers (HCPs), covalent organic frameworks (COFs), conjugated microporous polymers (CMPs), porous aromatic frameworks (PAFs), porous organic cages (PCs), silica gel, or any combination thereof.

In yet still further aspects, the antioxidant material can comprise ascorbic acid; alpha-, beta-, gamma-, or delta tocopherol; alpha-, beta-, gamma-, or delta-tocotrienol; polyphenols; beta-carotene; or a combination thereof.

Still further disclosed herein is a system for forming nitric oxide comprising: a source of NO₂; a vessel comprising the media of any of one of the examples herein; a patient interface coupled to the vessel and configured to deliver the nitric oxide to a patient.

Additional advantages will be set forth in part in the description that follows, and in part will be obvious from the description or can be learned by practice of the aspects described below. The advantages described below will be realized and attained by means of the chemical compositions, methods, and combinations thereof, particularly pointed out in the appended claims. It is to be understood that both the foregoing general description and the following detailed description are exemplary and explanatory only and are not restrictive of the invention, as claimed.

### DETAILED DESCRIPTION

The present invention can be understood more readily by reference to the following detailed description, examples, drawings, and claims, and their previous and following description. However, before the present articles, systems, and/or methods are disclosed and described, it is to be understood that this invention is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known aspect. To this end, those skilled in the relevant art will recognize and appreciate that many changes can be made to the various aspects of the invention described herein while still obtaining the beneficial results of the present invention. It will also be apparent that some of the desired benefits of the present invention can be obtained by selecting some of the features of the present invention without utilizing other features. Accordingly, those of ordinary skill in the pertinent art will recognize that many modifications and adaptations to the present invention are possible and may even be desirable in certain circumstances and are a part of the present invention. Thus, the following description is again provided as illustrative of the principles of the present invention and not in limitation thereof.

### Definitions

As used herein, the terms “optional” or “optionally” mean that the subsequently described event or circumstance can or cannot occur and that the description includes instances where said event or circumstance occurs and instances where it does not.

It is appreciated that certain features of the disclosure, which are, for clarity, described in the context of separate aspects, can also be provided in combination in a single aspect. Conversely, various features of the disclosure, which are, for brevity, described in the context of a single aspect, can also be provided separately or in any suitable subcombination.

As used in the description and the appended claims, the singular forms “a,” “an,” and “the” include plural referents unless the context clearly dictates otherwise. Thus, for example, a reference to “a single-use unit” includes not only one but also two or more such units, and a reference to “an apparatus” includes not only one but also two or more such apparatuses and the like.

Throughout the description and claims of this specification, the word “comprise” and other forms of the word, such as “comprising” and “comprises,” are open, non-limiting terms and mean “including but not limited to,” and are not intended to exclude, for example, other additives, segments, integers, or steps. Furthermore, it is to be understood that the terms “comprise,” “comprising,” and “comprises” as they relate to various aspects, elements, and features of the disclosed invention also include the more limited aspects of “consisting essentially of” and “consisting of.”

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate, effective amount will be readily determined by one of ordinary skill in the art.

Unless defined otherwise, all technical and scientific terms used herein have the same meaning as commonly understood by one of ordinary skill in the art to which this invention belongs. In this specification and in the claims which follow, reference will be made to a number of terms that shall be defined herein.

For the terms “for example” and “such as” and grammatical equivalences thereof, the phrase “and without limitation” is understood to follow unless explicitly stated otherwise. It is further understood that these phrases are used for explanatory purposes only. It is further understood that the term “exemplary,” as used herein, means “an example of” and is not intended to convey an indication of a preferred or ideal aspect.

The expressions “ambient temperature” and “room temperature” as used herein are understood in the art and refer generally to a temperature from 20° C. to 35° C.

All disclosed values also include values that fall within ±10% variation from the disclosed value unless otherwise indicated or inferred. In other words, if a range of 1 to 10 is disclosed, then a range of about 1 to about 10 is disclosed. In such aspects, it is understood that the amount or value in question can be the exact value or a value that provides equivalent results or effects as recited in the claims or taught herein. That is, amounts, sizes, formulations, parameters, and other quantities and characteristics include both exact values but also approximate, larger or smaller values as desired, reflecting tolerances, conversion factors, rounding, measurement error, and the like, and other factors known to those of skill in the art such that equivalent results or effects are obtained. In some circumstances, the value that provides equivalent results or effects cannot be reasonably determined. In general, an amount, size, formulation, parameter, or other quantity or characteristic is “about,” “approximate,” or “at or about,” whether or not expressly stated to be such. Where “about,” “approximate,” or “at or about” is used before a quantitative value, the parameter also includes the specific quantitative value itself unless expressly stated otherwise.

When a range is expressed, a further aspect includes from the one particular value and to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘x, y, z, or less’ and should be interpreted to include the specific ranges of ‘x,’ ‘y,’ ‘z,’ ‘about x,’ ‘about y,’ and ‘about z’ as well as the ranges of ‘less than x,’ ‘less than y, or ‘less than z,’ or ‘less than about x,’ ‘less than about y, and ‘less than about z.’ Likewise, the phrase’ x, y, z, or greater’ should be interpreted to include the specific ranges of ‘x,’ ‘y,’ ‘z,’‘about x,’ ‘about y,’ and ‘about z’ as well as the ranges of ‘greater than x,’ greater than y,’ ‘greater than z,’ or ‘greater than about x,’ greater than about y,’ ‘greater than about z.’ In addition, the phrase” ‘x’ to ‘y’,” where ‘x’ and y’ are numerical values, also includes “about x’ to about y’.”

Such a range format is used for convenience and brevity and, thus, should be interpreted flexibly to include not only the numerical values explicitly recited as the limits of the range but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5% but also include individual values (e.g., 1%, 2%, 3%, and 4%) and the sub-ranges (e.g., 0.5% to 1.1%; 5% to 2.4%; 0.5% to 3.2%, and 0.5% to 4.4%, and other possible sub-ranges) within the indicated range.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

In still further aspects, when the specific values are disclosed between two end values, it is understood that these end values can also be included.

In still further aspects, when the range is given, and exemplary values are provided, it is understood that any ranges can be formed between any exemplary values within the broadest range. For example, if individual numbers 1, 2, 3, 4, 5, 6, 7, etc. are disclosed, then the ranges 1-7, 2-7, 3-7, 4-7, 5-7, 6-7, 1-6, 1-5, 1-4, 1-3, 1-2, 2-6, 2-5, etc. are also disclosed.

References in the specification and concluding claims to parts by weight of a particular element or component in a composition denote the weight relationship between the element or component and any other elements or components in the composition or article for which a part by weight is expressed. Thus, in a mixture containing 2 parts by weight of component X and 5 parts by weight, components Y, X, and Y are present at a weight ratio of 2:5 and are present in such a ratio regardless of whether additional components are contained in the mixture.

A weight percent (wt. %) of a component, unless specifically stated to the contrary, is based on the total weight of the formulation or composition in which the component is included.

It will be understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”).

As used herein, the term “and/or” includes any and all combinations of one or more of the associated listed items.

It will be understood that although the terms “first,” “second,” etc., may be used herein to describe various elements, components, regions, layers, and/or sections. These elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of example aspects.

As used herein, the term “substantially” means that the subsequently described event or circumstance completely occurs or that the subsequently described event or circumstance generally, typically, or approximately occurs.

Still further, the term “substantially” can, in some aspects, refer to at least 90%, at least 95%, at least 99%, or 100% of the stated property, component, composition, or other condition for which substantially is used to characterize or otherwise quantify an amount.

In other aspects, as used herein, the term “substantially free,” when used in the context of a composition or component of a composition that is substantially absent, is intended to refer to an amount that is then 1% by weight, e.g., less than 0.5% by weight, less than 0.1% by weight, less than 0.05% by weight, or less than 0.01% by weight of the stated material, based on the total weight of the composition.

As used herein, “treating” and “treatment” generally refer to obtaining a desired pharmacological or physiological effect. The effect can be but does not necessarily have to be prophylactic in preventing or partially preventing a disease, symptom, or condition. The effect can be therapeutic regarding a partial or complete cure of a disease, condition, symptom, or adverse effect attributed to the disease, disorder, or condition. The term “treatment” as used herein can include any treatment of a disorder in a subject, particularly a human. It can include any one or more of the following: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease or its symptoms or conditions. The term “treatment,” as used herein, can refer to both therapeutic treatment alone, prophylactic treatment alone, or both therapeutic and prophylactic treatment. Those in need of treatment (i.e., subjects in need thereof) can include those already with the disorder or those in which the disorder is to be prevented. As used herein, the term “treating” can include inhibiting the disease, disorder, or condition, e.g., impeding its progress; and relieving the disease, disorder, or condition, e.g., causing regression of the disease, disorder, or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

Some aspects described herein relate to methods. It should be understood that such methods can be computer-implemented. That is, where the method or other events are described herein, it should be understood that they may be performed by a computing device having a processor and a memory. Memory of a computing device is also referred to as a non-transitory computer-readable medium, which can include instructions or computer code for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also referred to as code) may be those designed and constructed for a specific purpose or purpose. Examples of non-transitory computer-readable media include but are not limited to magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules, Read-Only Memory (ROM), Random-Access Memory (RAM) and/or the like. One or more processors can be communicatively coupled to the memory and operable to execute the code stored on the non-transitory processor-readable medium. Examples of processors include general purpose processors (e.g., CPUs), Graphical Processing Units, Field Programmable Gate Arrays (FPGAs), Application Specific Integrated Circuits (ASICs), Digital Signal Processor (DSPs), Programmable Logic Devices (PLDs), and the like. Examples of computer code include, but are not limited to, micro-code or micro-instructions, machine instructions, such as those produced by a compiler, code used to produce a web service, and files containing higher-level instructions that are executed by a computer using an interpreter. For example, aspects may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include but are not limited to, control signals, encrypted code, and compressed code.

While aspects of the present invention can be described and claimed in a particular statutory class, such as the system statutory class, this is for convenience only, and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to the arrangement of steps or operational flow, plain meaning derived from grammatical organization or punctuation, or the number or type of aspects described in the specification.

The present invention may be understood more readily by reference to the following detailed description of various aspects of the invention and the examples included therein.

### Device

Various systems and devices for generating nitric oxide (NO) are disclosed herein. Generally, NO is inhaled or otherwise delivered to a patient's lungs. Since NO is inhaled, much higher local doses can be achieved without concomitant vasodilation of the other blood vessels in the body. Accordingly, NO gas having a concentration of approximately 0.1 ppm to approximately 1000 ppm (e.g., 0.1, 0.5, 1, 5, 10, 40, 80, 100, 150, 200, 250, 300, 350, 400, 450, 500, 550, 600, 650, 700, 750, 800, 850, 900, 950, or 1000 ppm) may be delivered to a patient. Accordingly, high doses of NO may be used to prevent, reverse, or limit the progression of disorders, which can include, but are not limited to, acute pulmonary vasoconstriction, traumatic injury, aspiration or inhalation injury, fat embolism in the lung, acidosis, inflammation of the lung, adult respiratory distress syndrome, acute pulmonary edema, acute mountain sickness, post cardiac surgery acute pulmonary hypertension, persistent pulmonary hypertension of a newborn, perinatal aspiration syndrome, haline membrane disease, acute pulmonary thromboembolism, heparin-protamine reactions, sepsis, asthma, status asthmaticus, or hypoxia. NO can also be used to treat chronic pulmonary hypertension, bronchopulmonary dysplasia, chronic pulmonary thromboembolism, idiopathic pulmonary hypertension, primary pulmonary hypertension, or chronic hypoxia.

Currently, approved devices and methods for delivering inhaled NO gas require complex and heavy equipment. NO gas is stored in heavy gas bottles with nitrogen and no traces of oxygen. NO gas is mixed with air or oxygen with specialized injectors and complex ventilators, and the mixing process is monitored with equipment having sensitive microprocessors and electronics. All this equipment is required in order to ensure that NO is not oxidized into nitrogen dioxide (NO₂) during the mixing process since NO₂ is highly toxic. However, this equipment is not conducive to use in a non-medical facility setting since the size, cost, complexity, and safety issues restrict the operation of this equipment to highly-trained professionals in a medical facility.

Some of the devices for forming nitric oxide from NO₂ are disclosed in U.S. Pat. Nos. 8,607,785, 8,944,049, 9,604,028, 10,926,054, 11,744,978, the contents of which are incorporated herein in their whole entirety.

In contrast, the devices and systems disclosed herein do not require the storage of nitric oxide in heavy gas bottles. The devices disclosed herein allow the formation of nitric oxide from nitrogen dioxide on demand, when a predetermined amount of nitric oxide is needed to be delivered to a patient.

In certain aspects, disclosed herein is a device comprising a media configured to convert nitrogen dioxide into nitric oxide, wherein the media comprises: (a) a support material having a specific surface area of 350 to 5000 m²/g; and (b) an antioxidant material.

In such aspects, the specific surface area of the support material is 350 to 5000 m²/g, including exemplary values of 400 m²/g, 500 m²/g, 600 m²/g, 700 m²/g, 800 m²/g, 900 m²/g, 1000 m²/g, 1250 m²/g, 1500 m²/g, 1750 m²/g, 2000 m²/g, 2250 m²/g, 2500 m²/g, 2750 m²/g, 3000 m²/g, 3250 m²/g, 3500 m²/g, 3750 m²/g, 4000 m²/g, 4250 m²/g, 4500 m²/g, 4750 m²/g, and 4990 m²/g. It is understood that the specific surface area can be in any range formed between any two foregoing values. For example, and without limitations, the specific surface area can be 350 to 4500 m²/g, 400 to 5000 m²/g, 400 to 4000 m²/g, 500 to 5000 m²/g, or 500 to 4000 m²/g, or 500 to 3000 m²/g or 500 to 2000 m²/g and so on.

In still further aspects, the support material can be a macroporous, mesoporous, or microporous material. In certain aspects, the support material is macroporous. In other aspects, the support material is mesoporous, in still further aspects, the support material is microporous. In yet still further aspects, the support material can be a mixture of macroporous portions and/or mesoporous portions, and/or microporous portions. In still further aspects, the support material can comprise a continuous polymer phase permeated by a continuous pore phase.

It is understood that the term “continuous” in the context of polymer phase or pore phase generally refers to a phase such that all points within the phase are directly connected so that for any two points within a continuous phase, there exists a path which connects the two points without leaving the phase.

In still further aspects, the support material disclosed herein can have a pore size of 1 angstrom to 1000 angstroms, including exemplary values of 2 angstroms, 5 angstroms, 10 angstroms, 15 angstroms, 20 angstroms, 25 angstroms, 30 angstroms, 35 angstroms, 40 angstroms, 50 angstroms, 60 angstroms, 70 angstroms, 80 angstroms, 90 angstroms, 100 angstroms, 200 angstroms, 300 angstroms, 400 angstroms, 500 angstroms, 600 angstroms, 700 angstroms, 800 angstroms, and 900 angstroms. It is understood that the pore size can be in any range formed between any two foregoing values. For example, and without limitations, the pore size can be 1 to 1000 angstroms, or 1 to 500 angstroms, or 1 to 200 angstroms, or 1 to 100 angstroms, or 1 to 50 angstroms, or 1 to 40 angstroms, or 1 to 30 angstroms, and so on.

In still further aspects, the support material has a pore size of 1 angstrom to 100 angstrom, including exemplary values of 2 angstroms, 5 angstroms, 10 angstroms, 15 angstroms, 20 angstroms, 25 angstroms, 30 angstroms, 35 angstroms, 40 angstroms, 50 angstroms, 60 angstroms, 70 angstroms, 80 angstroms, and 90 angstroms. It is understood that the pore size can be in any range formed between any two foregoing values. For example, and without limitations, the pore size can be 1 to 100 angstroms, or 1 to 80 angstroms, or 1 to 70 angstroms, or 1 to 50 angstroms, or 1 to 40 angstroms, or 1 to 30 angstroms, and so on.

In still further aspects, the support material has a pore size of 1 angstrom to 50 angstrom, including exemplary values of 2 angstroms, 3 angstroms, 4 angstroms, 5 angstroms, 6 angstroms, 7 angstroms, 8 angstroms, 9 angstroms, 10 angstroms, 11 angstroms, 12 angstroms, 13 angstroms, 14 angstroms, 15 angstroms, 16 angstroms, 17 angstroms, 18 angstroms, 19 angstroms, 20 angstroms, 21 angstroms, 22 angstroms, 23 angstroms, 24 angstroms, 25 angstroms, 26 angstroms, 27 angstroms, 28 angstroms, 29 angstroms, 30 angstroms, 31 angstroms, 32 angstroms, 33 angstroms, 34 angstroms, 35 angstroms, 36 angstroms, 37 angstroms, 38 angstroms, 39 angstroms, 40 angstroms, 41 angstroms, 42 angstroms, 43 angstroms, 44 angstroms, 45 angstroms, 46 angstroms, 47 angstroms, 48 angstroms, and 49 angstroms. In still further aspects, a pore size of less than 40 angstroms. It is understood that the pore size can be in any range formed between any two foregoing values. For example, and without limitations, the pore size can be 1 to 50 angstroms, or 1 to 45 angstroms, or 1 to 40 angstroms, or 1 to 35 angstroms, or 1 to 30 angstroms, or 1 to 25 angstroms, or 1 to 20 angstroms, or 1 to 15 angstroms, or 1 to 10 angstroms, and so on.

In still further aspects, the support material comprises a molecular sieve, a metal-organic framework (MOF), natural zeolites, synthetic zeolites, polymers of intrinsic microporosity (PIMs), hyper-crosslinked microporous polymers (HCPs), covalent organic frameworks (COFs), conjugated microporous polymers (CMPs), porous aromatic frameworks (PAFs), porous organic cages (PCs), silica gel, or any combination thereof.

In certain aspects, the support can comprise natural and/or synthetic zeolites. In yet other aspects, the support material can comprise a molecular sieve. In yet other aspects, the support material can comprise silica gel. In still further aspects, the support material can comprise MOFs. For example, and without limitations, MOFs can be Al-based, Ti-based, Zr-based, Ni-based, Co-based, Cu-based, and so on, and any combination thereof.

In still further aspects, the support material can comprise polymers of intrinsic microporosity (PIMs). It is understood that any known PIMs can be utilized. For example, and without limitations, any PIMs disclosed in “Polymer of Intrinsic Microporosity” by N. B. McKeown (International Scholarly Research Network, volume 2012, article ID 513986, 16 pages, doi: 10.5402/2012/513986), U.S. Pat. No. 8,623,928, WO2003000774 and/or WO2005012397, each of which is hereby incorporated by reference herein in its entirety for its teachings on polymers of intrinsic microporosity.

In still further aspects, the support material can comprise hyper-crosslinked microporous polymers (HCPs). Any known in the art HCPs can be used. For example, and without limitations, hyper-crosslinked microporous polymers include Tetraphenyl anthraquinone-based HCP, Binaphthol-based HCP, and A porous organic polymer containing triazine and carbazole moieties. Some additional and unlimiting examples can be found in Polymers, 2017, 9(12), 651 by R. Castaldo et al. in “Microporous Hyper-Crosslinked polystyrenes and nanocomposites with high adsorption properties: A Review,” which is hereby incorporated by reference herein in its entirety.

In still further aspects, the support material can comprise covalent organic frameworks (COFs). Any known in the art COFs can be used. Some unlimiting examples of such materials and methods of making the same are described in Giant, 6, 2021, by H. R. Abuzeid et al. in “Covalent organic frameworks: Design principles, synthetic strategies, and diverse applications,” or Polymers, 2021, 13(6), 970, by T. F. Machado et al. in “Covalent Organic Frameworks: Synthesis, Properties and Applications—An Overview,” each of which is hereby incorporated by reference herein in its entirety for its teachings on covalent organic frameworks.

In still further aspects, the support material can comprise conjugated microporous polymers (CMPs). Any known in the art CMPs can be used. Some unlimited examples can be found in Chemical Reviews, 2020, 120, 2171-2214 by J-Sing M. Lee et al. in “Advances in Conjugated Microporous Polymers,” which is hereby incorporated by reference herein in its entirety.

In still further aspects, the support material can comprise porous aromatic frameworks (PAFs). Any known in the art PAFs can be used. Some unlimited examples can be found in Chemical Reviews, 2020, 120, 16, 8934-8986 by Y. Tian et al. in “Porous Aromatic Frameworks,” which is hereby incorporated by reference herein in its entirety.

In still further aspects, the support material can comprise porous organic cages (PCs). Any known in the art PCs can be used. Some unlimited examples can be found in Chemical Reviews, 2023, 123, 8, 4602-4634 by X. Yang et al. in “Porous Organic Cages,” which is hereby incorporated by reference herein in its entirety.

In still further aspects, the support material can comprise an amount of water. In certain aspects, water is present as moisture. In yet other aspects, water molecules can be incorporated within the pores of the support materials or on the surface of the support material.

In still further aspects, the media has a moisture content of greater than 0 to less than 100 wt % based on the total weight of the support material, including exemplary values of 0.01 wt %, 0.05 wt %, 0.1 wt %, 0.5 wt %, 1 wt %, 2.5 wt %, 5 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, 80 wt %, 85 wt %, 90 wt %, 95 wt %, and 99 wt % based on the total weight of the support material. It is understood that the moisture content can be in any range formed between any two foregoing values. For example, and without limitations, the moisture content can be 0.01 to 50 wt %, or 0.1 to 50 wt %, or 1 to 50 wt %, or 5 to 50 wt %, or 10 to 50 wt %, or 1 to 25 wt %, or 0.1 to 30 wt %, or 0.5 to 30 wt %, or 1 to 30 wt %, and so on. Yet in still further aspects, the moisture content can be 0.01 to 80 wt %, or 0.1 to 70 wt %, or 1 to 60 wt %, or 5 to 90 wt %, or 10 to 80 wt %, or 1 to 75 wt %, or 0.1 to 60 wt %, or 0.5 to 70 wt %, or 25 to 60 wt %, and so on. In still further aspects, the water present in the support material can be present in an effective amount to form the desired concentration of nitric oxide.

In still further aspects, the antioxidant material can be provided as an aqueous solution. In such exemplary and unlimiting aspects, the support material is then impregnated by the antioxidant material. In still further aspects, the impregnated with antioxidant material the support material is dried to remove undesirable amount of water. In still further aspects, at least some of the water can remain. It is understood that the amount of water can be determined by the skilled practitioner based on the specific support material, antioxidant, the desired concentration of nitric oxide to be formed, and the like. It is understood that the terms impregnated and embedded as described herein can be used interchangeably.

In still further aspects, the antioxidant material is embedded (or impregnated) within at least a portion of the support material. Yet in still further aspects, the antioxidant material is substantially uniformly embedded within the support material. In yet still further aspects, the antioxidant material coats at least a portion of the support material. Yet in still further aspects, the antioxidant material substantially uniformly coats the support material. Yet in still further aspects, the antioxidant material comprises ascorbic acid; alpha-, beta-, gamma-, or delta tocopherol; alpha-, beta-, gamma-, or delta-tocotrienol; polyphenols; beta-carotene; or a combination thereof.

It is understood that the support materials disclosed herein allow for a more efficient interaction between the antioxidant and N₂O₄ and/or NO₂ to produce nitric oxide.

An exemplary reaction between the nitrogen dioxide and the media disclosed herein is shown below.

a. 6NO₂(gas)+3H₂O (liq.)→3HNO₃ (liq.)+3HNO₂ (liq.) Eq. 1a

b. 3HNO₂(liq.)→HNO₃ (liq.)+2NO (gas)+H2O (liq.) Eq. 1b

Yet in other aspects, the NO can be formed according to Eq. 2a-2b:

C₆H₈O₆+NO₂=>C₆H₆O₆+NO+H₂O Eq. 2a

3NO₂+H₂O=>2HNO₃+NO Eq. 2b

In still further aspects, the antioxidant material is present in an amount of greater than 0 wt % to less than 100 wt % based on the total weight of the media, including exemplary values of 0.01 wt %, 0.05 wt %, 0.1 wt %, 0.5 wt %, 1 wt %, 2.5 wt %, 5 wt %, 10 wt %, 15 wt %, 20 wt %, 25 wt %, 30 wt %, 35 wt %, 40 wt %, 45 wt %, 50 wt %, 55 wt %, 60 wt %, 65 wt %, 70 wt %, 75 wt %, 80 wt %, 85 wt %, 90 wt %, 95 wt %, and 99 wt % based on the total weight of the media. It is understood that the antioxidant material can be in any range formed between any two foregoing values. For example, and without limitations, the antioxidant material can be greater than 0 to 50 wt %, greater than 0 to 45 wt %, greater than 0 to 40 wt %, or greater than 0 to 35 wt %, or greater than 0 to 30 wt %, or greater than 0 to 25 wt %, or greater than 0 to 20 wt %, or greater than 0 to 15 wt %, or greater than 0 to 10 wt %, and so on.

In still further aspects, the antioxidant material is present in an effective amount to form a predetermined amount of nitric oxide. In yet still, in further aspects, the support material is present in an effective amount to form a predetermined amount of nitric oxide.

In still further aspects, the media disclosed herein can comprise a polymeric material. In such aspects, the polymeric material can be different from the polymeric material present in the support material. In still further aspects, the polymeric material can be a thermoplastic material. In yet still further aspects, the polymeric material can comprise polyethylene, polypropylene, polyamide, polyurethane, polystyrene, or any combination thereof.

Any of the disclosed above antioxidants and/or support materials can be used to form the nitric oxide in the desired amount.

For example and without limitations, the media can comprise: (a) the support material comprising: 0 to 50 wt % of a polymeric material and greater than 0 wt % to 70 wt % of silica gel, wherein the wt % is calculated based on the total mass of the media; and (b) the antioxidant material comprising an ascorbic acid in an amount of greater than 0 to 50 wt %, wherein the wt % is calculated based on the total mass of the media.

In such exemplary aspects, the polymeric material can be present in an amount of 0 to 50 wt %, 0 to 45 wt %, 0 to 40 wt %, 0 to 35 wt %, 0 to 30 wt %, 0 to 25 wt %, 0 to 20 wt %, 0 to 15 wt %, 0 to 10 wt %, 0 to 5 wt %, 0 to 1 wt %, 1 to 50 wt %, 5 to 50 wt %, 10 to 50 wt %, 15 to 50 wt %, 20 to 50 wt %, 25 to 50 wt %, 30 to 50 wt %, 35 to 50 wt %, and so on. In still further aspects, the polymeric material is, for example, polyethylene. In still further aspects, the polymeric material can be present in an amount of 25 to 35 wt %, 25 wt % to 30 wt %.

In still further aspects, the support material can be a silica gel present in an amount of and greater than 0 wt % to 70 wt %, greater than 0 wt % to 65 wt %, and greater than 0 wt % to 60 wt %, and greater than 0 wt % to 55 wt %, and greater than 0 wt % to 50 wt %, and greater than 0 wt % to 45 wt %, and greater than 0 wt % to 40 wt %, and greater than 0 wt % to 35 wt %, and greater than 0 wt % to 30 wt %, and greater than 0 wt % to 25 wt %, or and greater than 0 wt % to 20 wt %. In yet other aspects, the silica gel present in an amount of and greater than 0 wt % to 70 wt %, 5 wt % to 70 wt %, 10 wt % to 70 wt %, 15 wt % to 70 wt %, 20 wt % to 70 wt %, 35 wt % to 70 wt %, 40 0 wt % to 70 wt %, and so on. In still further aspects, the silica gel can be present 35 wt % to 55 wt %, 35 wt % to 50 wt %, 35 wt % to 45 wt %, 35 wt % to 40 wt %.

In still further exemplary and unlimiting aspects, antioxidant material can comprise an ascorbic acid in an amount greater than 0 to 50 wt %, wherein the wt % is calculated based on the total mass of the media. In such exemplary aspects, the ascorbic acid can be present in an amount of 0 to 50 wt %, 0 to 45 wt %, 0 to 40 wt %, 0 to 35 wt %, 0 to 30 wt %, 0 to 25 wt %, 0 to 20 wt %, 0 to 15 wt %, 0 to 10 wt %, 0 to 5 wt %, 0 to 1 wt %, 1 to 50 wt %, 5 to 50 wt %, 10 to 50 wt %, 15 to 50 wt %, 20 to 50 wt %, 25 to 50 wt %, 30 to 50 wt %, 35 to 50 wt %, and so on. In still further aspects, the ascorbic acid can be present in an amount 10 to 40 wt %, 10 to 35 wt %, 10 to 30 wt %, 10 to 30 wt %, 10 to 25 wt %, 10 to 20 wt %, 10 to 15 wt %, and so on. It is understood, however, that any of the disclosed antioxidants can be present instead or in addition to ascorbic acid.

In still further aspects, the media comprises: a) the support material comprising: 0 to less than 100 wt % of a polymeric material and greater than 0 wt % to less than 100 wt % of a natural or synthetic zeolite, wherein the wt % is calculated based on the total mass of the media; and b) the antioxidant material comprising an ascorbic acid in an amount of greater than 0 to 50 wt %, wherein the wt % is calculated based on the total mass of the media.

In such exemplary aspects, the polymeric material can be present in an amount of 0 to less than 100 wt %, 0 to 95 wt %, 0 to 90 wt %, 0 to 85 wt %, 0 to 80 wt %, 0 to 75 wt %, 0 to 70 wt %, 0 to 65 wt %, 0 to 60 wt %, 0 to 55 wt %, 0 to 50 wt %, 0 to 45 wt %, 0 to 40 wt %, 0 to 45 wt %, 0 to 40 wt %, 0 to 45 wt %, 0 to 40 wt %, 0 to 15 wt %. In yet still further aspects, the polymeric material in this example can be in an amount of 1 to less than 100 wt %, 5 to less than 100 wt %, 10 to less than 100 wt %, 15 to less than 100 wt %, 20 to less than 100 wt %, 25 to less than 100 wt %, 30 to less than 100 wt %, 35 to less than 100 wt %, 40 to less than 100 wt %, 45 to less than 100 wt %, 50 to less than 100 wt %, or 60 to less than 100 wt %, and so on. In still further aspects, the polymeric material can be present in an amount of 40 to 70 wt %, 40 to 65 wt %, 40 to 60 wt %, 40 to 55 wt %, 40 to 50 wt %, or 40 to 45 wt %.

In certain aspects, the support material can be a zeolite present in an amount of and greater than 0 wt % to 70 wt %, greater than 0 wt % to 65 wt %, and greater than 0 wt % to 60 wt %, and greater than 0 wt % to 55 wt %, and greater than 0 wt % to 50 wt %, and greater than 0 wt % to 45 wt %, and greater than 0 wt % to 40 wt %, and greater than 0 wt % to 35 wt %, and greater than 0 wt % to 30 wt %, and greater than 0 wt % to 25 wt %, or and greater than 0 wt % to 20 wt %. In yet other aspects, the zeolite can be present in an amount of and greater than 0 wt % to 70 wt %, 5 wt % to 70 wt %, 10 wt % to 70 wt %, 15 wt % to 70 wt %, 20 wt % to 70 wt %, 35 wt % to 70 wt %, 40 0 wt % to 70 wt %, and so on. In still further aspects, the zeolite can be present in an amount of 20 wt % to 40 wt %, 25 wt % to 40 wt %, 30 wt % to 40 wt %, or 35 wt % to 40 wt %.

In still further exemplary and unlimiting aspects, antioxidant material can comprise an ascorbic acid in an amount greater than 0 to 50 wt %, wherein the wt % is calculated based on the total mass of the media. In such exemplary aspects, the ascorbic acid can be present in an amount of 0 to 50 wt %, 0 to 45 wt %, 0 to 40 wt %, 0 to 35 wt %, 0 to 30 wt %, 0 to 25 wt %, 0 to 20 wt %, 0 to 15 wt %, 0 to 10 wt %, 0 to 5 wt %, 0 to 1 wt %, 1 to 50 wt %, 5 to 50 wt %, 10 to 50 wt %, 15 to 50 wt %, 20 to 50 wt %, 25 to 50 wt %, 30 to 50 wt %, 35 to 50 wt %, and so on. In still further aspects, the ascorbic acid can be present in an amount 10 to 40 wt %, 10 to 35 wt %, 10 to 30 wt %, 10 to 30 wt %, 10 to 25 wt %, 10 to 20 wt %, 10 to 15 wt %, and so on. In still further aspects, the ascorbic acid can be present in an amount of 10 to 25 wt %, 10 to 20 wt %, or 10 to 15 wt %. It is understood, however, that any of the disclosed antioxidants can be present instead or in addition to ascorbic acid.

In still further aspects, the media can comprise: a) the support material comprising: 0 to less than 100 wt % of a polymeric material and greater than 0 wt % to less than 100 wt % of a metal organic framework, wherein the wt % is calculated based on the total mass of the media; and b) the antioxidant material comprising an ascorbic acid in an amount of greater than 0 to 50 wt %, wherein the wt % is calculated based on the total mass of the media.

In still further exemplary aspects, the support material can be a metal organic framework present in an amount of and greater than 0 wt % to 70 wt %, greater than 0 wt % to 65 wt %, and greater than 0 wt % to 60 wt %, and greater than 0 wt % to 55 wt %, and greater than 0 wt % to 50 wt %, and greater than 0 wt % to 45 wt %, and greater than 0 wt % to 40 wt %, and greater than 0 wt % to 35 wt %, and greater than 0 wt % to 30 wt %, and greater than 0 wt % to 25 wt %, or and greater than 0 wt % to 20 wt %. In yet other aspects, the metal organic framework is present in an amount of and greater than 0 wt % to 70 wt %, 5 wt % to 70 wt %, 10 wt % to 70 wt %, 15 wt % to 70 wt %, 20 wt % to 70 wt %, 35 wt % to 70 wt %, 40 0 wt % to 70 wt %, and so on.

In such examples, the polymeric material and antioxidant can be any amount, as disclosed above.

In still further aspects, the media is disposed within a vessel. In some exemplary and unlimiting aspects, the media is formed within the vessel. In yet other aspects, the media can conform to the vessel's dimensions and shape. In still further aspects, the media is formed into a shape that conforms to the vessel.

In still further aspects, the media is present in a granular form that fills the vessel's dimensions in an effective amount relative to the volume of the vessel.

In still further aspects, the media is present in a liquid form that fills the vessel's dimensions in an effective amount relative to the volume of the vessel.

In yet still further aspects, the media is present in a gel form that fills the vessel's dimensions in an effective amount relative to the volume of the vessel.

In still further aspects, the media is formed into a shape that conforms to the vessel.

In still further aspects, the media can also comprise a gel. In certain aspects, the gel can be a hydrogel. In still further aspects, the gel can comprise the support material and the antioxidant dispersed within it. In still further aspects, the media can comprise a suspension. In still further aspects, the media is present in a solid form, a granular form, a gel, a liquid form, or any combination thereof.

In certain exemplary and unlimiting aspects, the vessel containing the disclosed herein media is a pressure vessel. It is understood that the term “pressure vessel” as used herein refers to any vessel capable of containing (a liquid and/or gas and/or fluid and/or gel or solid) and/or withstanding pressure up to 1000 psi, up to 900 psi, up to 800 psi, up to 700 psi, up to 600 psi, or up to 500 psi. In still further aspects, the pressure vessel as described herein is capable of containing (a liquid and/or gas and/or fluid) and/or withstanding pressure of equal to or greater than 14 psi to 1000 psi, including exemplary values of 15 psi, 50 psi, 100 psi, 150 psi, 200 psi, 250 psi, 300 psi, 350 psi, 400 psi, 450 psi, 500 psi, 550 psi, 600 psi, 650 psi, 700 psi, 750 psi, 800 psi, 850 psi, 900 psi, and 950 psi. It is understood that any ranges between any two foregoing values can be formed. For example, and without limitations, the pressure vessel as described herein is capable of containing a liquid and/or gas and/or fluid and/or withstanding pressure of 15 psi to 600 psi, 15 psi to 500 psi, or 15 psi to 400 psi, or 100 psi to 900 psi, and so on. It is understood that the first chamber is made of a material capable of withstanding the disclosed pressures and chemical conditions under which the chamber operates. In certain exemplary and limiting aspects, the first vessel can be made of stainless steel, aluminum, inductive metals, alloys thereof, thermally conductive polymers, or any combination thereof.

In still further aspects, the media is in fluid communication with a source of NO₂. It is understood that the source of NO₂ can be any source. For example, in some aspects, the source of NO₂ can be liquid N₂O₄. In other aspects, the source of NO₂ can be N₂O₄ incorporated into a matrix, for example, a gel. In other aspects, it is understood that N₂O₄ can be in equilibrium with NO₂. In still further aspects, the source of NO₂ can be a gas tank comprising NO₂, and so on.

In still further aspects, the media comprises a fluidic pathway configured to transfer formed nitric oxide to a patient.

Also disclosed herein is a system for forming nitric oxide comprising: a source of NO₂; a vessel comprising any of the disclosed herein media a patient interface coupled to the vessel and configured to deliver the nitric oxide to a patient. In still further aspects, the system comprises one or more humidifiers that are in communication with the vessel. In still further aspects, the system comprises a control unit and one or more sensors configured to measure an amount of formed nitric oxide and/or an amount of nitrogen dioxide. The system can further comprise inerting chambers configured to inert residual or leaked nitrogen dioxide.

While various aspects have been described above, it should be understood that they have been presented by way of example only and not limitation. Furthermore, although various aspects have been described as having particular features and/or combinations of components, other aspects possibly have a combination of any features and/or components from any of the aspects where appropriate, as well as additional features and/or components.

Where methods described above indicate certain events occurring in a certain order, the ordering of certain events may be modified. Additionally, certain of the events may be performed concurrently in a parallel process, when possible, as well as performed sequentially as described above. Although various aspects have been described as having particular features and/or combinations of components, other aspects possibly have a combination of any features and/or components from any of the aspects where appropriate.

### EXEMPLARY ASPECTS

Exemplary Aspect 1. A device comprising: a media configured to convert nitrogen dioxide into nitric oxide, wherein the media comprises: (a) a support material having a specific surface area of 350 to 5000 m²/g; and (b) an antioxidant material.

Exemplary Aspect 2. The device of any of one of the examples herein, particularly Exemplary Aspect 1, wherein the support material is a macroporous, mesoporous, or microporous material.

Exemplary Aspect 3. The device of any of one of the examples herein, particularly Exemplary Aspect 1 or 2, wherein the support material comprises a molecular sieve, a metal-organic framework (MOF), natural zeolites, synthetic zeolites, polymers of intrinsic microporosity (PIMs), hyper-crosslinked microporous polymers (HCPs), covalent organic frameworks (COFs), conjugated microporous polymers (CMPs), porous aromatic frameworks (PAFs), porous organic cages (PCs), silica gel, or any combination thereof.

Exemplary Aspect 4. The device of any of one of the examples herein, particularly Exemplary Aspect 3, wherein the support material further comprises an amount of water.

Exemplary Aspect 5. The device of any of one of the examples herein, particularly Exemplary Aspects 1-4, wherein the support material has a pore size of 1 angstrom to 1000 angstroms.

Exemplary Aspect 6. The device of any of one of the examples herein, particularly Exemplary Aspects 1-5, wherein the support material has a pore size of 1 angstrom to 100 angstrom.

Exemplary Aspect 7. The device of any of one of the examples herein, particularly Exemplary Aspects 1-6, wherein the support material has a pore size of 1 angstrom to 50 angstrom.

Exemplary Aspect 8. The device of any of one of the examples herein, particularly Exemplary Aspects 1-7, wherein the support material has a pore size of less than 40 angstroms.

Exemplary Aspect 9. The device of any of one of the examples herein, particularly Exemplary Aspects 1-8, wherein the antioxidant material is embedded within at least a portion of the support material.

Exemplary Aspect 10. The device of any of one of the examples herein, particularly Exemplary Aspects 1-9, wherein the antioxidant material is substantially uniformly embedded within the support material.

Exemplary Aspect 11. The device of any of one of the examples herein, particularly Exemplary Aspects 1-10, wherein the antioxidant material coats at least a portion of the support material.

Exemplary Aspect 12. The device of any of one of the examples herein, particularly Exemplary Aspects 1-11, wherein the antioxidant material substantially uniformly coats the support material.

Exemplary Aspect 13. The device of any of one of the examples herein, particularly Exemplary Aspects 1-12, wherein the antioxidant material comprises ascorbic acid; alpha-, beta-, gamma-, or delta tocopherol; alpha-, beta-, gamma-, or delta-tocotrienol; polyphenols; beta-carotene; or a combination thereof.

Exemplary Aspect 14. The device of any of the examples herein, particularly Exemplary Aspects 1-13, wherein the media further comprises a polymeric material.

Exemplary Aspect 15. The device of any of one of the examples herein, particularly Exemplary Aspect 14, wherein the polymeric material is a thermoplastic resin.

Exemplary Aspect 16. The device of any of one of the examples herein, particularly Exemplary Aspects 1-15, wherein the media further comprises a gel.

Exemplary Aspect 17. The device of any of one of the examples herein, particularly Exemplary Aspects 1-16, wherein the media is present in a solid form, a granular form, a gel, a liquid form, or any combination thereof.

Exemplary Aspect 18. The device of any of one of the examples herein, particularly Exemplary Aspects 1-17, wherein the media has a moisture content of greater than 0 to less than 100% based on the total weight of the support material.

Exemplary Aspect 19. The device of any of one of the examples herein, particularly Exemplary Aspects 1-18, wherein the antioxidant material is present in an amount of greater than 0 wt % to less than 100 wt % based on the total weight of the media.

Exemplary Aspect 20. The device of any of one of the examples herein, particularly Exemplary Aspects 1-19, wherein the antioxidant material is present in an amount greater than 0 wt % to 50 wt % based on the total weight of the media.

Exemplary Aspect 21. The device of any of one of the examples herein, particularly Exemplary Aspects 1-20, wherein the antioxidant material is present in an amount greater than 0 wt % to 25 wt % based on the total weight of the media.

Exemplary Aspect 22. The device of any of one of the examples herein, particularly Exemplary Aspects 1-21, wherein the antioxidant material is present in an effective amount to form a predetermined amount of nitric oxide.

Exemplary Aspect 23. The device of any of one of the examples herein, particularly Exemplary Aspects 1-22, wherein the support material is present in an effective amount to form a predetermined amount of nitric oxide.

Exemplary Aspect 24. The device of any of one of the examples herein, particularly Exemplary Aspects 1-23, wherein the media is disposed within a vessel.

Exemplary Aspect 25. The device of any of one of the examples herein, particularly Exemplary Aspect 24, wherein the media is formed within the vessel.

Exemplary Aspect 26. The device of any of one of the examples herein, particularly Exemplary Aspect 25, wherein the media conforms to the vessel's dimensions.

Exemplary Aspect 27. The device of any of one of the examples herein, particularly Exemplary Aspect 24, wherein the media is present in a granular form that fills the vessel's dimensions in an effective amount relative to the volume of the vessel.

Exemplary Aspect 28. The device of any of one of the examples herein, particularly Exemplary Aspect 24, wherein the media is present in a liquid form that fills the vessel's dimensions in an effective amount relative to the volume of the vessel.

Exemplary Aspect 29. The device of any of one of the examples herein, particularly Exemplary Aspect 24, wherein the media is present in a gel form that fills the vessel's dimensions in an effective amount relative to the volume of the vessel.

Exemplary Aspect 30. The device of any of one of the examples herein, particularly Exemplary Aspects 1-29, wherein the media comprises: a) the support material comprising: 0 to 50 wt % of a polymeric material and greater than 0 wt % to 70 wt % of silica gel, wherein the wt % is calculated based on the total mass of the media; and b) the antioxidant material comprising an ascorbic acid in an amount of greater than 0 to 50 wt %, wherein the wt % is calculated based on the total mass of the media.

Exemplary Aspect 31. The device of any of one of the examples herein, particularly Exemplary Aspect 30, wherein the media comprises a) the support material comprising: 25 to 35 wt % of a polymeric material and 35 wt % to 55 wt % of silica gel, wherein the wt % is calculated based on the total mass of the media; and b) the antioxidant material comprising an ascorbic acid in an amount 10 to 40 wt %, wherein the wt % is calculated based on the total mass of the media.

Exemplary Aspect 32. The device of any of one of the examples herein, particularly Exemplary Aspect 30 or 31, wherein the polymeric material is polyethylene.

Exemplary Aspect 33. The device of any of one of the examples herein, particularly Exemplary Aspects 30-32, wherein the media is formed into a shape conforming shape of the vessel.

Exemplary Aspect 34. The device of any of one of the examples herein, particularly Exemplary Aspects 1-29, wherein the media comprises: a) the support material comprising: 0 to less than 100 wt % of a polymeric material and greater than 0 wt % to less than 100 wt % of a natural or synthetic zeolite, wherein the wt % is calculated based on the total mass of the media; and b) the antioxidant material comprising an ascorbic acid in an amount of greater than 0 to 50 wt %, wherein the wt % is calculated based on the total mass of the media.

Exemplary Aspect 35. The device of any of one of the examples herein, particularly Exemplary Aspect 34, wherein the media comprises a) the support material comprising: 40 to 70 wt % of a polymeric material and 20 wt % to 40 wt % of a natural or synthetic zeolite, wherein the wt % is calculated based on the total mass of the media; and b) the antioxidant material comprising an ascorbic acid in an amount 10 to 25 wt %, wherein the wt % is calculated based on the total mass of the media.

Exemplary Aspect 36. The device of any of one of the examples herein, particularly Exemplary Aspect 34 or 35, wherein the polymeric material is polyethylene.

Exemplary Aspect 37. The device of any of one of the examples herein, particularly Exemplary Aspects 34-36, wherein the media is formed into a shape conforming shape of the vessel.

Exemplary Aspect 38. The device of any of one of the examples herein, particularly Exemplary Aspects 1-29, wherein the media comprises: a) the support material comprising: 0 to less than 100 wt % of a polymeric material and greater than 0 wt % to less than 100 wt % of a metal organic framework, wherein the wt % is calculated based on the total mass of the media; and b) the antioxidant material comprising an ascorbic acid in an amount of greater than 0 to 50 wt %, wherein the wt % is calculated based on the total mass of the media.

Exemplary Aspect 39. The device of any of one of the examples herein, particularly Exemplary Aspect 38, wherein the polymeric material is polyethylene.

Exemplary Aspect 40. The device of any of one of the examples herein, particularly Exemplary Aspect 38 or 39, wherein the media is formed into a shape conforming shape of the vessel.

Exemplary Aspect 41. The device of any of one of the examples herein, particularly Exemplary Aspects 1-40, wherein the media is in fluid communication with a source of NO₂.

Exemplary Aspect 42. The device of any of one of the examples herein, particularly Exemplary Aspects 1-41, wherein the media comprises a fluidic pathway configured to transfer formed nitric oxide to a patient.

Exemplary Aspect 43. A system for forming nitric oxide comprising: a source of NO₂; a vessel comprising the media of any of one of the examples herein, particularly Exemplary Aspects 1-42; a patient interface coupled to the vessel and configured to deliver the nitric oxide to a patient.

Exemplary Aspect 44. The system of any of one of the examples herein, particularly Exemplary Aspect 43, further comprising one or more humidifiers that are in communication with the vessel.

What is claimed is:

1. A device comprising:
a media configured to convert nitrogen dioxide into nitric oxide, wherein the media comprises:
a) a support material having a specific surface area of 350 to 5000 m²/g; wherein the support material is a macroporous, mesoporous, or microporous material; and
b) an antioxidant material, wherein the antioxidant material is present in an amount of greater than 0 wt % to less than 100 wt % based on a total weight of the media.

2. The device of claim 1, wherein the support material comprises a molecular sieve, a metal-organic framework (MOF), natural zeolites, synthetic zeolites, polymers of intrinsic microporosity (PIMs), hyper-crosslinked microporous polymers (HCPs), covalent organic frameworks (COFs), conjugated microporous polymers (CMPs), porous aromatic frameworks (PAFs), porous organic cages (PCs), silica gel, or any combination thereof.

3. The device of claim 2, wherein the support material further comprises an amount of water.

4. The device of claim 1, wherein the support material has a pore size of 1 angstrom to 1000 angstroms; and/or wherein the support material has a pore size of 1 angstrom to 100 angstrom, and/or wherein the support material has a pore size of 1 angstrom to 50 angstrom; and/or wherein the support material has a pore size of less than 40 angstroms.

5. The device of claim 1, wherein the antioxidant material:
i) is embedded within at least a portion of the support material; and/or
ii) is substantially uniformly embedded within the support material; and/or
iii) coats at least a portion of the support material; and/or
iv) substantially uniformly coats the support material.

6. The device of claim 1, wherein the antioxidant material comprises ascorbic acid; alpha-, beta-, gamma-, or delta tocopherol; alpha-, beta-, gamma-, or delta-tocotrienol; polyphenols; beta-carotene; or a combination thereof.

7. The device of claim 1, wherein the media further comprises a polymeric material and/or wherein the media further comprises a gel.

8. The device of claim 1, wherein the media is present in a solid form, a granular form, a gel, a liquid form, or any combination thereof.

9. The device of claim 1, wherein the media has a moisture content of greater than 0 to less than 100 wt % based on the total weight of the support material.

10. The device of claim 1, wherein the antioxidant material is present in an amount greater than 0 wt % to 50 wt % based on the total weight of the media.

11. The device of claim 1, wherein the antioxidant material is present in an effective amount to form a predetermined amount of nitric oxide, and/or wherein the support material is present in an effective amount to form a predetermined amount of nitric oxide.

12. The device of claim 1, wherein the media is disposed within a vessel.

13. The device of claim 12, wherein the media is formed within the vessel and wherein the media conforms to vessel's dimensions.

14. The device of claim 13, wherein
i) the media is present in a granular form that fills the vessel's dimensions in an effective amount relative to a volume of the vessel, or
ii) the media is present in a liquid form that fills the vessel's dimensions in an effective amount relative to the volume of the vessel, or
iii) the media is present in a gel form that fills the vessel's dimensions in an effective amount relative to the volume of the vessel.

15. The device of claim 1, wherein the media comprises:
a) the support material comprising: 0 to 50 wt % of a polymeric material and greater than 0 wt % to 70 wt % of silica gel, wherein the wt % is calculated based on a total mass of the media; and
b) antioxidant material comprising an ascorbic acid in an amount of greater than 0 to 50 wt %, wherein the wt % is calculated based on the total mass of the media.

16. The device of claim 15, wherein the media comprises:
a) the support material comprising: 25 to 35 wt % of a polymeric material and 35 wt % to 55 wt % of silica gel, wherein the wt % is calculated based on the total mass of the media; and
b) antioxidant material comprising an ascorbic acid in an amount of 10 to 40 wt %, wherein the wt % is calculated based on the total mass of the media.

17. The device of claim 1, wherein the media comprises:
a) the support material comprising: 0 to less than 100 wt % of a polymeric material and greater than 0 wt % to less than 100 wt % of a natural or synthetic zeolite, wherein the wt % is calculated based on a total mass of the media; and
b) the antioxidant material comprising an ascorbic acid in an amount of greater than 0 to 50 wt %, wherein the wt % is calculated based on the total mass of the media.

18. The device of claim 17, wherein the media comprises:
a) the support material comprising: 40 to 70 wt % of a polymeric material and 20 wt % to 40 wt % of a natural or synthetic zeolite, wherein the wt % is calculated based on a total mass of the media; and
b) the antioxidant material comprising an ascorbic acid in an amount of 10 to 25 wt %, wherein the wt % is calculated based on the total mass of the media.

19. The device of claim 1, wherein the media comprises:
a) the support material comprising: 0 to less than 100 wt % of a polymeric material and greater than 0 wt % to less than 100 wt % of a metal organic framework, wherein the wt % is calculated based on a total mass of the media; and
b) the antioxidant material comprising an ascorbic acid in an amount of greater than 0 to 50 wt %, wherein the wt % is calculated based on the total mass of the media.

20. The device of claim 1, wherein the media is in fluid communication with a source of NO₂, and/or wherein the media comprises a fluidic pathway configured to transfer formed nitric oxide to a patient.
